(19) United States
(12) Patent Application Publication
(10) Pub. No.: US 2022/0226065 A1
(43) Pub. Date: Jul. 21, 2022

(54) Camera System for Healthcare

(71) Applicant: CinVivo, Redwood City, CA (US)

(72) Inventor: Bernard A. Hausen, Redwood City, CA (US)

(21) Appl. No.: 17/579,498

(22) Filed: Jan. 19, 2022

Related U.S. Application Data
(60) Provisional application No. 63/139,397, filed on Jan. 20, 2021.

(51) Int. Cl. A61B 90/00; H04N 5/225; H04N 5/232

(52) U.S. Cl. CPC A61B 90/361; A61B 90/37; A61B 2090/371; H04N 5/23206; H04N 5/2251

(57) ABSTRACT

A camera system for medical use may include an image signal processor; at least one head-mounted camera assembly that includes a camera module, where the camera module includes an inertial sensor connected to the image signal processor, a time-of-flight sensor connected to the image signal processor, an image sensor connected to the image signal processor, and a liquid lens positioned relative to the image sensor such that the image sensor collects light from the liquid lens; and a hub connected to the image signal processor.

### CROSS-REFERENCE TO RELATED APPLICATIONS

This application claims the benefit of priority to U.S. Provisional Application No. 63/139,937, filed on Jan. 20, 2021, which is hereby incorporated by reference in its entirety.

### FIELD OF THE INVENTION

The invention generally relates to cameras, and more particularly to the use of a camera system for healthcare.

### BACKGROUND

The use of endoscopes or other cameras insertable into the body is necessary for some types of minimally-invasive (also called endoscopic) surgery. Such minimally-invasive surgery may be performed directly by a surgeon, or may be performed using a surgical robot such as the daVinci® Surgical System of Intuitive Surgical (Sunnyvale, Calif.). An example of the type of minimally-invasive surgery that requires a camera to be inserted into the body to view the surgical site is totally endoscopic coronary artery bypass graft (TECAB) surgery. Whether a surgeon uses tools directly inserted by hand into the thoracic cavity to place the graft, or uses a surgical robot, a camera must view the site of attachment of the graft to the coronary artery or the aorta. The camera outputs video to one or more monitors, allowing the surgeon to view that video and control tools or the surgical robot to properly place and attach the graft. In addition, other healthcare professionals in the vicinity, such as but not limited to anesthesiologists, nurses, medtechs, and vendor representatives, can view the video and remain engaged with the procedure. Thus, even though minimally-invasive surgery is performed through small ports in the patient's body, multiple people in the operating room or other surgical location can easily view the procedure and its progress.

Paradoxically, open surgery is often difficult for anyone other than the surgeon and at most two attending nurses or other professionals to watch. The incisions in the patient may be large. The chest of the patient may be open. However, the surgeon is positioned adjacent to the patient, as is an attending nurse. Their bodies block the view by others. Further, even without the surgeon or attending nurse standing adjacent to the patient, it can be difficult to see the surgical site even a few feet away. Further, the anesthesiologist is typically seated, as are other professionals in the operating room, and their elevation is not high enough to see into the surgical site. As a result, it can be difficult for people other than the surgeon and the attending nurse to remain engaged with the procedure, resulting in less-than-optimal care for the patient.

Cameras are known that mount to the surgical light handle commonly used in operating rooms. However, such cameras typically need to be frequently repositioned in use, which is inconvenient. The surgeon's head or hands often obscure the surgical field. The video quality is often insufficient due to the distance between the camera and the surgical field. Typically, such cameras do not provide audio, either. Large mobile camera systems on wheels/casters are also known, in which cameras are placed on long swivelable arms. Such cameras have the same issues as the light-handle-mounted cameras described above. In addition, such mobile camera systems have a large footprint in a confined operating environment, which can make their use challenging. Further, such devices may fall into the category of capital equipment and typically are expensive, restricting their adoption at hospitals.

Simple head cameras are available that may be used in an operating room environment. However, such cameras are typically produced by lighting manufacturers with little camera expertise. Such cameras typically have a wired connection to a computer or other device, which limits mobility of the wearer. Their sensors and lenses are off-the-shelf, and are not adapted to the views, colors and illumination of the surgical field. Further, such cameras do not typically include audio capability, nor do they include software or hardware to remotely control, cast or stream video.

Further, deficiencies in camera systems are present in other healthcare environments, inside and outside the hospital. In environments such as catheterization labs, treatment rooms, diagnosis rooms, emergency rooms, accident sites, and locations outside of a hospital or healthcare building, cameras may not be standard equipment, or even equipment that is available to any user. As a result, in many healthcare environments, a live video feed and/or a video record of diagnosis is not even possible to obtain, much less store. Such a record, which is not currently obtained or stored, may be useful for educating medical students and professionals, and for documenting proper procedure was followed in order to limit any potential liability.

Thus, there is an unmet need for camera systems for a complete spectrum of healthcare uses. Further, there is an unmet need for camera systems that overcome the disadvantages of current camera systems and smart camera systems that may be used in environments such as an operating room.

### SUMMARY OF THE INVENTION

A camera system for medical use may include an image signal processor; at least one head-mounted camera assembly that includes a camera module, where the camera module includes an inertial sensor connected to the image signal processor, a time-of-flight sensor connected to the image signal processor, an image sensor connected to the image signal processor, and a liquid lens positioned relative to the image sensor such that the image sensor collects light from the liquid lens; and a hub connected to the image signal processor.

A method for utilizing a camera system in conjunction with providing healthcare to a patient by at least one user may include placing a head-mounted camera assembly onto at least one user; acquiring video with each head-mounted camera assembly; stabilizing acquired video in the head-mounted camera assembly; transmitting stabilized video from each head-mounted camera assembly to a hub; receiving stabilized video at the hub; and controlling video output from the hub.

### BRIEF DESCRIPTION OF THE DRAWINGS

FIG. 1A is a side view of a camera assembly including a light module and a camera module.

FIG. 1B is a first perspective view of a camera assembly including a light module and a camera module.

FIG. 1C is a second perspective view of a camera assembly including a light module and a camera module.

FIG. 2A is a perspective view of the light module and the camera module of FIG. 1.

FIG. 2B is a side view of the light module and the camera module of FIG. 2A.

FIG. 2C is a perspective view of the light module and the camera module of FIG. 2A, where the light module includes a swappable lens.

FIG. 2D is another perspective view of the light module and the camera module of FIG. 2A, where the light module includes a swappable lens.

FIG. 2E is a rear view of the light module and the camera module of FIG. 2A.

FIG. 3 is a schematic view of the camera module architecture of FIG. 1.

FIG. 4A is a perspective view of a user wearing the camera assembly of FIG. 1 attached to a headband, a transceiver at the user's neck area, and a battery.

FIG. 4B is a side view of the user of FIG. 4A.

FIG. 4C is a rear view of the user of FIG. 4A.

FIG. 4D is a perspective view of a user wearing the camera assembly of FIG. 1 attached to a headband, a transceiver at the user's waist, and a battery.

FIG. 5A is a front view of a transceiver.

FIG. 5B is a top view of the transceiver of FIG. 5A.

FIG. 5C is a perspective view of the transceiver of FIG. 5A.

FIG. 5D is a rear view of the transceiver of FIG. 5A.

FIG. 6 shows the arrangement of FIGS. 6A and 6B relative to one another.

FIG. 6A is a first page of a schematic view of a camera assembly of FIG. 1 and a transceiver.

FIG. 6B is a second page of a schematic view of a camera assembly of FIG. 1 and a transceiver.

FIG. 7 is a schematic view of a hub with a data connection to the transceiver of FIG. 3.

FIG. 7A is a perspective view of a hub with a data connection to the transceiver of FIG. 3, showing also a monitor and tablet.

FIG. 8 is a perspective view of a camera attached to a surgical tool.

FIG. 9 is a side view of a camera attached to a surgical tool.

FIG. 10 is a schematic view of a location in which the camera assembly of FIG. 1 and the hub of FIGS. 7-7A may be utilized.

FIG. 11 is a schematic view of the location of FIG. 10, where that location is an operating room.

The use of the same reference symbols in different figures indicates similar or identical items.

### DETAILED DESCRIPTION

System

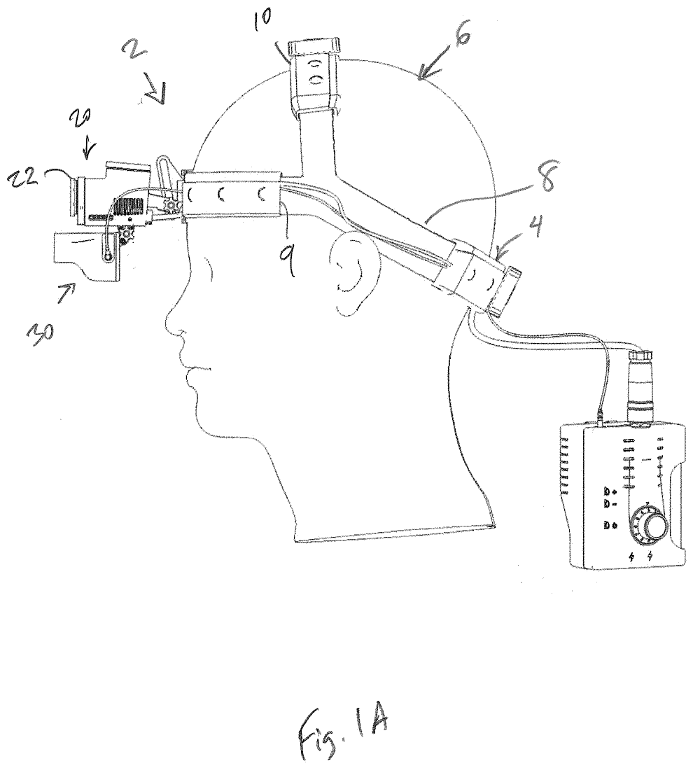

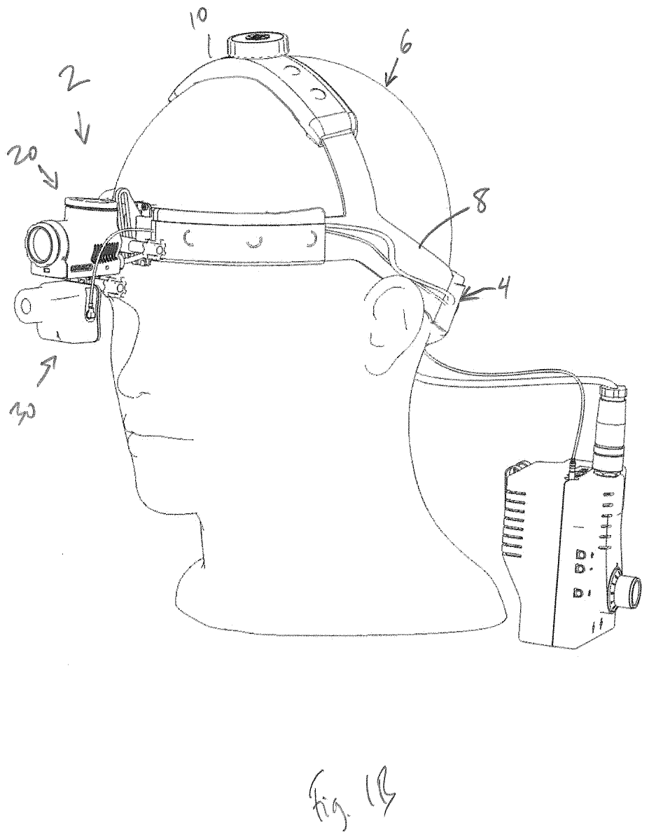

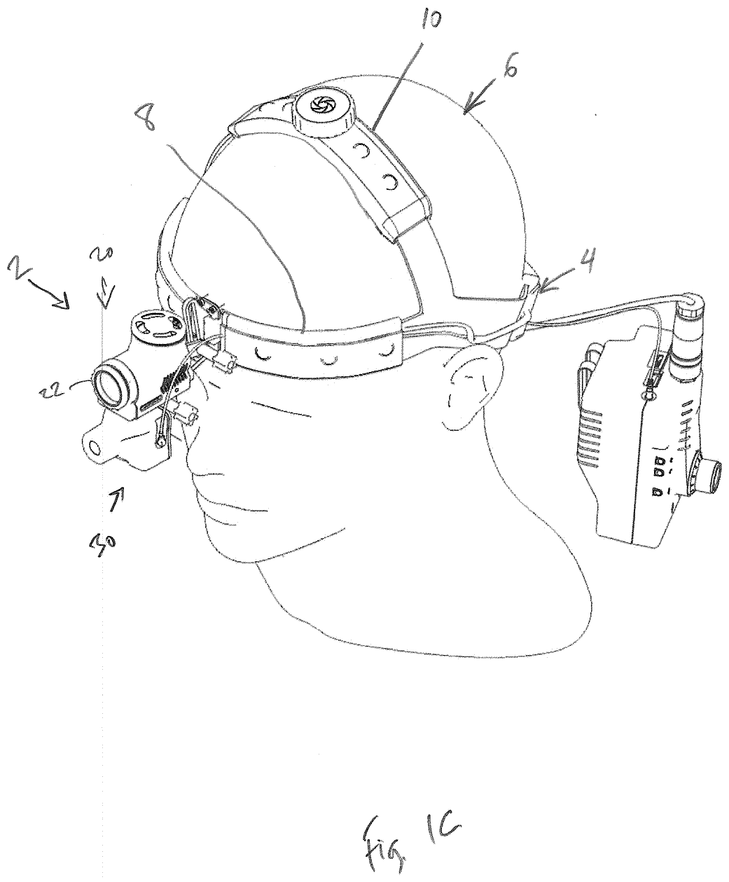

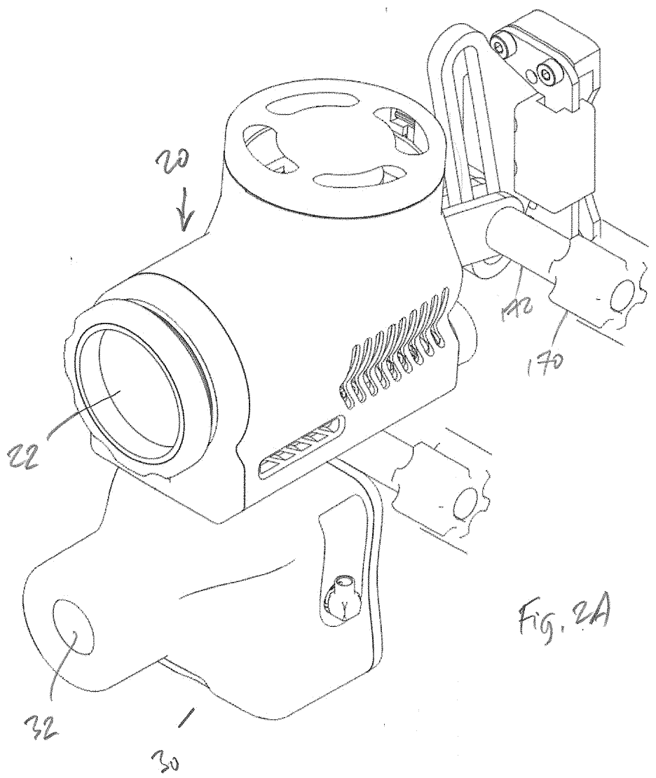

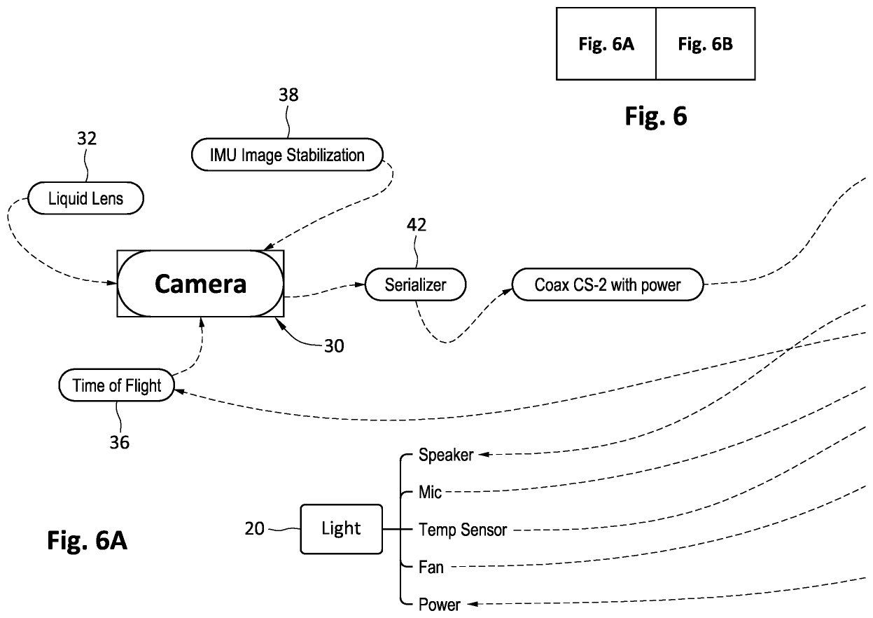

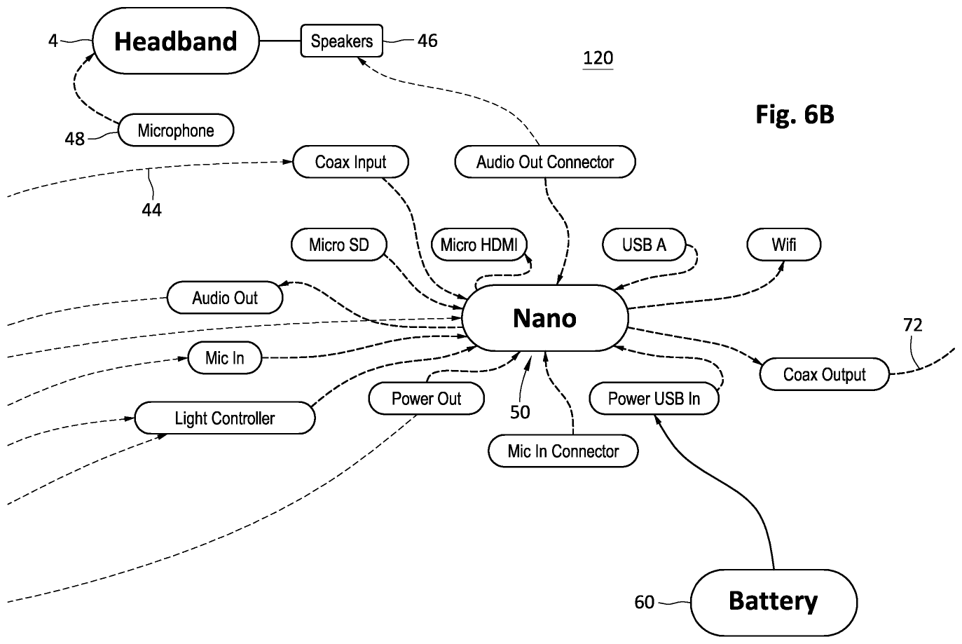

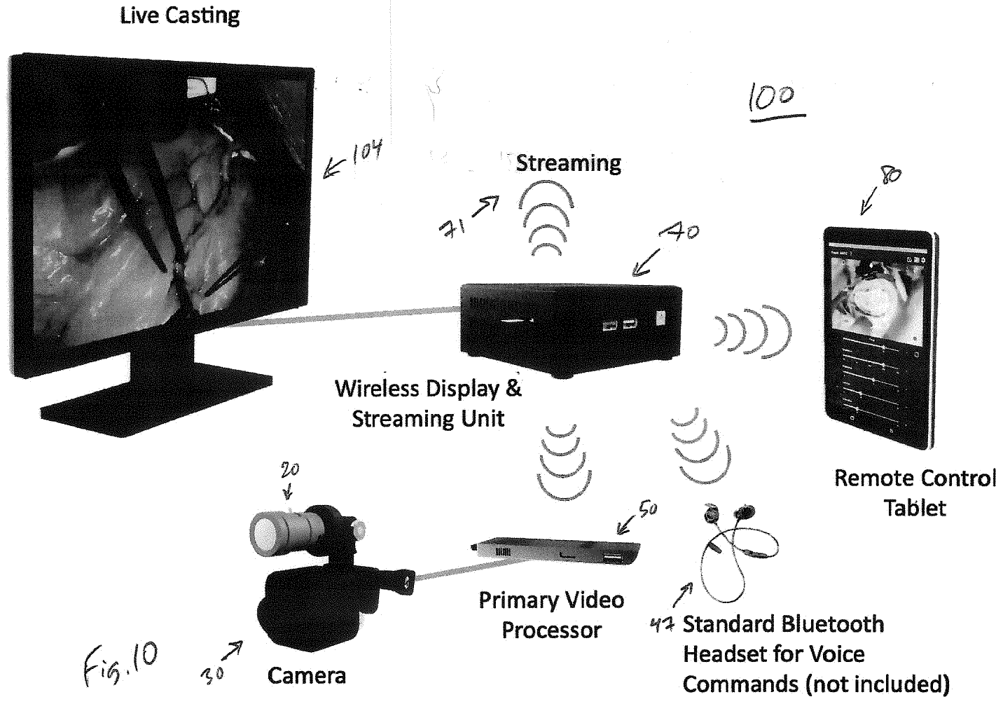

Referring to FIGS. 1A-1C, an exemplary camera assembly 2 is shown. The camera assembly 2 includes a head mount 4 configured to fit onto a user's head 6. The head mount 4 may include a strap 8 that may be adjustable by the user, in any suitable manner, to fit comfortably around his or her head. The head mount 4 optionally may include a top strap 10 that extends from one side of the strap 8, over the top of the user's head 6, to the other side of the strap 8. The top strap 10 assists in load-bearing, and may support a majority of the weight of the camera assembly 2 by contact with the top of the user's head 6. The top strap 10 may be adjustable by the user, in any suitable manner, to fit comfortably onto his or her head. The strap 8 and top strap 10 may be partially or completely elastic, or substantially inelastic. The strap 8 and top strap 10 may be fabricated from any suitable material. Referring also to FIG. 6, the camera assembly 2 may include speakers 46 and a microphone 48. As another example, referring also to FIG. 10, the speakers 46 and microphone 48 may be part of a standard headset 47. The headset 47 may use a wireless connection, such as one meeting the BLUETOOTH® (Bluetooth SIG, Kirkland, Wash.) standard, to connect to the transceiver 50 as described below.

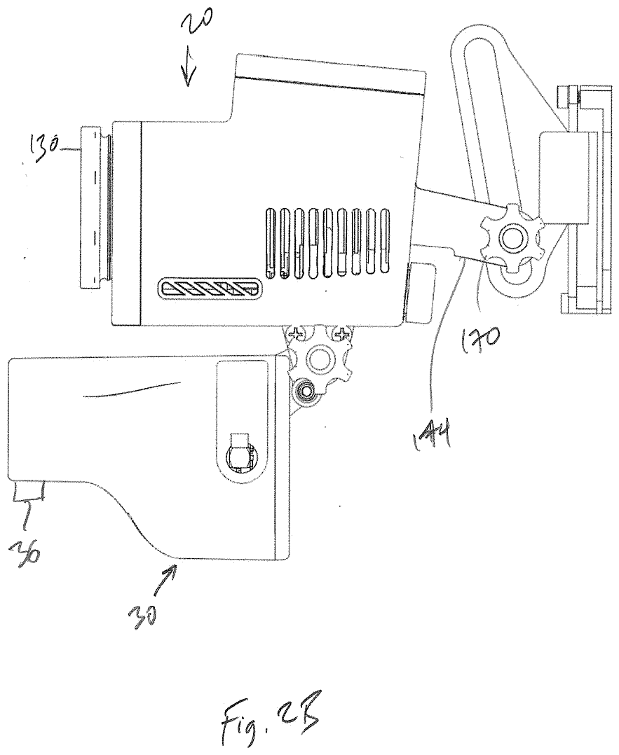

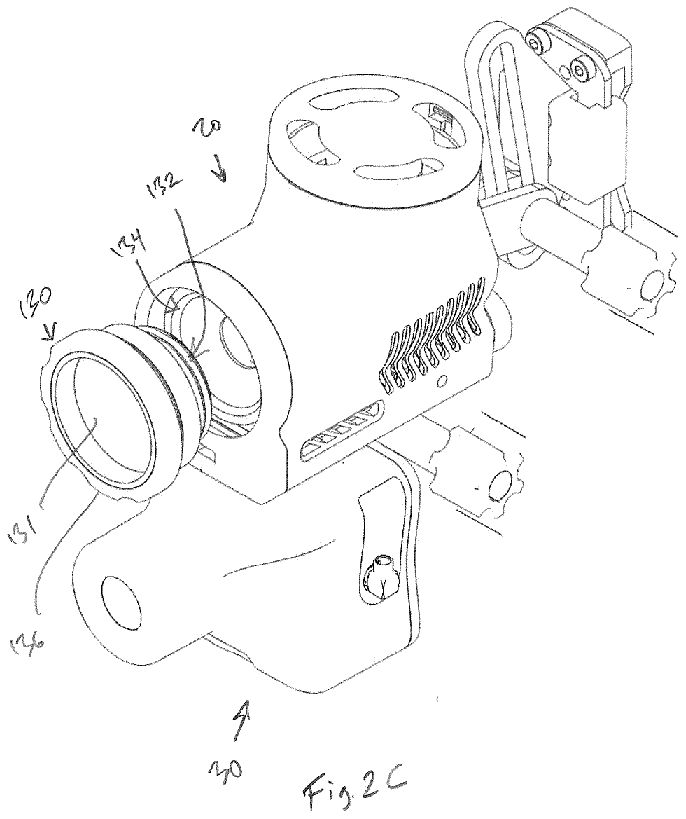

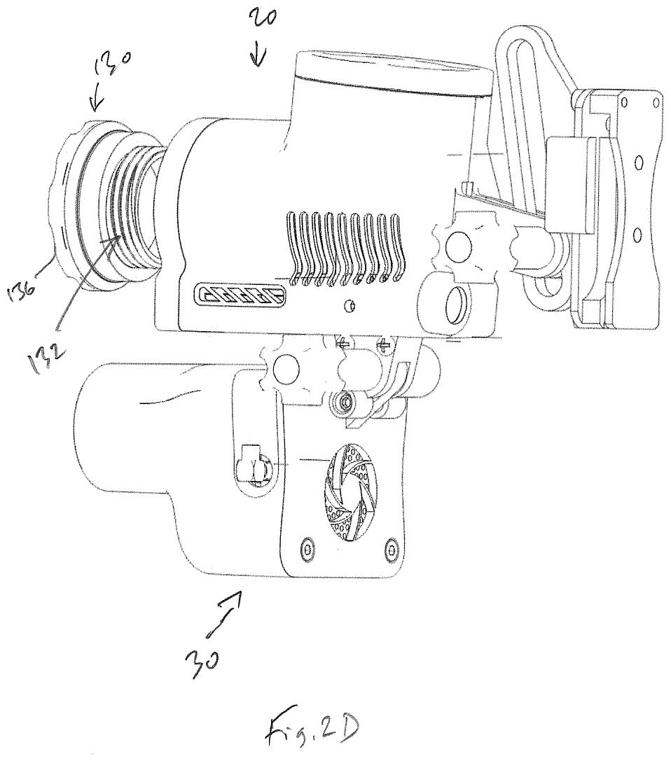

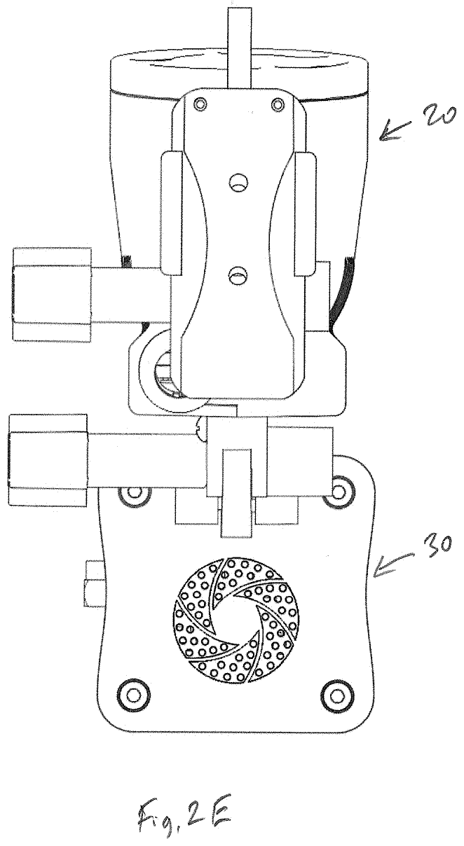

Referring also to FIGS. 2A-2E, the camera assembly 2 includes a light module 20. The light module 20 may be attached to the head mount 4. The attachment between the light module 20 and the head mount 4 may be accomplished in any suitable manner. According to one embodiment, the light module 20 may be substantially fixed to the head mount 4. According to another embodiment, the light module 20 may be movable relative to the head mount 4, such as via a swivel joint or other connection allowing for movement of the light module 20 relative to the head mount 4. Where the light module 20 is movable relative to the head mount 4, the light module 20 may be lockable relative to the head mount 4 after the light module 20 has been moved to a desired position. The light module 20 is configured to emit light through an opening 22. The source of light from the light module may be one or more light-emitting diodes (LEDs), one or more laser diodes (LDs), one or more injection laser diodes (ILDs), one or more diode lasers, one or more xenon high-intensity discharge (HID) lamps, one or more halogen lamps, or any other suitable lighting source that is capable of being worn on a head mount 4 for the duration of a surgical procedure. Advantageously, the light module 20 produces light with a color rendering index (CRI) over 80; even more advantageously, the light module 20 produces light with a CRI over 90. A lens and/or diffuser may be positioned in, on or in proximity to the opening 22, if desired.

The light module 20 may be configured to receive a swappable lens 130. In existing surgical lights or surgical lamps, a light shines onto a spot in the surgical field, and the diameter of that spot is adjusted using an iris through which light passes before it passes through a lens. Changing the diameter of the iris changes the amount of light that can pass through the iris, and thus through the lens, accordingly; decreasing the diameter of the iris reduces the amount of light that passes through the lens, and increasing the diameter of the iris increases the amount of light that passes through the lens. Thus, to tighten the spot diameter that is illuminated in the surgical field, the iris is tightened, and less light passes through the lens. The illuminance (lux) reaching the spot in the surgical field is equal to luminous flux (lumens) divided by area; the decrease in luminous flux from the reduction of the diameter of the iris is greater than the decrease in area of the spot diameter that is illuminated in the surgical field, such that the illuminance is decreased.

The use of swappable lenses 130 eliminates that problem with prior art surgical lighting. Different swappable lenses 130 may be utilized with the light module 20, where each swappable lens 130 is associated with a different fixed spot diameter in the surgical field. The lens element 131 of each swappable lens 130 may be glass, or may be fabricated from any other suitable material. The swappable lenses 130 may be threaded with threads 132 that are configured to be received by light module threads 134. According to other embodiments, the swappable lenses 130 may be detachably connected to the light module 20 in any other suitable manner and with any other suitable mechanism, such as by a quick disconnect. The swappable lenses 130 optionally include a grippable ring 136 defined at an end or another location thereon. The grippable ring 136 may be rubberized or treated in a manner to increase friction when grasped by a user, to allow for convenient unscrewing of a swappable lens 130 and screwing in of another swappable lens 130. When a user wishes to decrease the spot diameter illuminated in the surgical field, the user detaches the swappable lens 130 currently attached to the light module 20, and attaches a different swappable lens associated with that smaller spot diameter. No iris is utilized, and as a result, the amount of light passing through the swappable lens 130 is unchanged. Consequently, because the same amount of light passes through the swappable lens 130 to a smaller spot diameter, the illuminance of that spot diameter in the surgical field is increased.

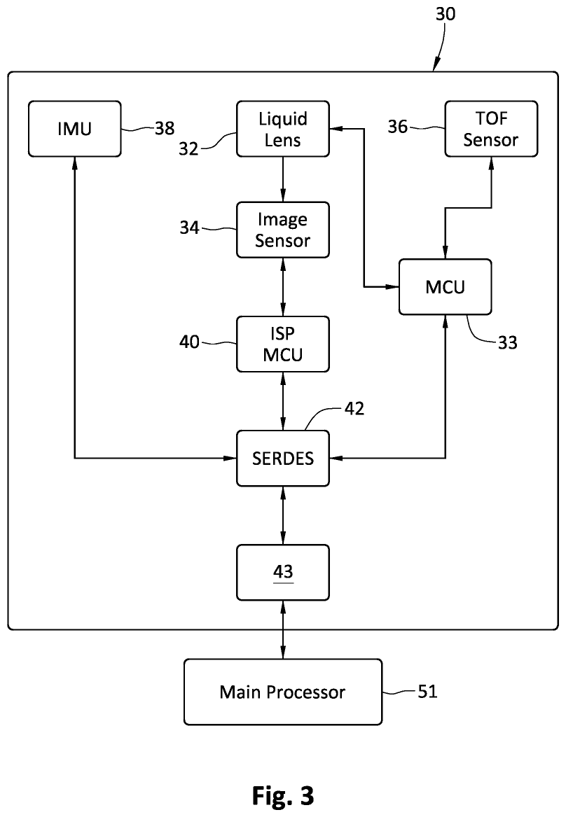

Referring also to FIGS. 2A-2E and 3, the camera assembly 2 includes a camera module 30. According to some embodiments, the camera module 30 may be attached to the light module 20 The attachment between the light module 20 and the camera module 30 may be accomplished in any suitable manner. According to one embodiment, the light module 20 may be substantially fixed to the camera module 30. According to another embodiment, the camera module 30 may be movable relative to the light module 20, such as via a swivel joint or other connection allowing for movement of the camera module 30 relative to the light module 20. Where the camera module 30 is movable relative to the light module 20, the camera module 30 may be lockable relative to the light module 20 after the camera module 30 has been moved to a desired position.

According to other embodiments, the camera module 30 may be attached to the head mount 4. The attachment between the camera module 30 and the head mount 4 may be accomplished in any suitable manner. According to one embodiment, the camera module 30 may be substantially fixed to the head mount 4. According to another embodiment, the camera module 30 may be movable relative to the head mount 4, such as via a swivel joint or other connection allowing for movement of the camera module 30 relative to the head mount 4. Where the camera module 30 is movable relative to the head mount 4, the camera module 30 may be lockable relative to the head mount 4 after the camera module 30 has been moved to a desired position. In such embodiments, the light module 20 may be attached directly to the camera module 30 in a manner such as described above with regard to the connection of the camera module 30 directly to the light module 20.

According to other embodiments, the light module 20 and camera module 30 may be integrated into a single module.

As seen in FIG. 1, the light module 20 and camera module 30 are in close proximity to one another. In this way, the illumination provided by the light module 20 is generally aligned with the field of view of the camera module 30. Fixing the light module 20 and camera module 30 together may be advantageous, in that the alignment of the light module 20 and the camera module 30 may be preset and maintained at the preset. Allowing the camera module 30 to be moved relative to the light module 20 allows the user to change the alignment as desired.

Referring also to FIGS. 2A and 3, according to some embodiments, the camera module 30 includes a liquid lens 32. Like traditional optical lenses made from glass, a liquid lens 32 is a single optical element that includes an optical liquid material that can change its shape. The focal length of the liquid lens 32 is changed by controlling the radius of curvature and/or the index of refraction of the optical liquid material. This change in radius is electronically controlled, and rapidly changes on the order of milliseconds. Technologies ranging from electrowetting to shape changing polymers to acousto-optic tuning may be used to control the radius of curvature and index of refraction of the liquid lens 32; any suitable method may be used to focus the liquid lens 32.

An image sensor 34 is placed in the camera relative to the liquid lens 32 to collect light from the liquid lens 32. Optionally, one or more intermediate lenses (not shown) may be placed in the optical path between the liquid lens 32 and the image sensor 34 in a multi-element structure.

According to some embodiments, the camera module 30 includes a time-of-flight sensor 36 in proximity to the liquid lens 32. According to some embodiments, the time-of-flight sensor 36 emits intermittent pulses of light, which may be generated by an LED, a laser, or any other suitable source. The time between pulses of light may be regular, or may be irregular and linked to motion of the camera module 30. The light emitted by the time-of-flight sensor 36 may be in the infrared range of wavelengths, according to some embodiments; according to other embodiments, the light emitted by the time-of-flight sensor 36 may be in a different range of wavelengths. The light emitted by the time-of-flight sensor 36 is reflected by objects in the field of view of the camera module 30, and a portion of that reflected light is received by the time-of-flight sensor 36. The time between emission of the light pulse by the time-of-flight sensor 36 and the sensing by the time-of-flight sensor 36 of light reflected from that light pulse by an objects illuminated by the time-of-flight sensor 36 allows the distance between the time-of-flight sensor 36 and those objects to be calculated.

According to other embodiments, the time-of-flight sensor 36 emits light continuously. The amplitude of the emitted light is modulated, creating a light source of a sinusoidal form at a known and controlled frequency. The reflected light is phase-shifted, and the time-of-flight sensor 36 determines the phase shift of the reflected light to calculate the distance between the time-of-flight sensor 36 and objects illuminated by the time-of-flight sensor 36.

According to other embodiments, the time-of-flight sensor 36 may be a lidar device. Regardless of which embodiment of the time-of-flight sensor 36 is utilized, the time-of-flight sensor 36 provides fast and precise measurements of the distance between the time-of-flight sensor 36 and the objects illuminated thereby—in this application, those objects are structures in a patient's body within the surgical field. The use of a time-of-flight sensor 36 in conjunction with a liquid lens 32 in the camera module 30 allows for very fast and accurate focusing on the area of the surgical field where the surgeon or other user is looking. The focusing provided by the combination of the time-of-flights sensor 36 and the liquid lens 32 may be continuous or near-continuous, maintaining the image of the objects in the field of view of the image sensor 34 in focus or very close to focus. Data from the time-of-flight sensor 36 may be routed through a microcontroller 33 and then transmitted to the liquid lens 32. According to some embodiments, the microcontroller 33 may process the range data received from the time-of-flight sensor 36 and then transmit focusing instructions directly to the liquid lens 32. According to other embodiments, one or more other components of the camera system 2 may perform such processing.

According to some embodiments, the camera module 30 may include an inertial sensor 38. The inertial sensor 38 may include one or more accelerometers. Advantageously, the inertial sensor 38 includes accelerometers that measure acceleration along each of three orthogonal axes. The inertial sensor 38 may include one or more gyroscopes, such as but not limited to MEMS gyroscopes. Advantageously, the inertial sensor 38 includes gyroscopes that measure rotation about each of three orthogonal axes.

According to some embodiments, the camera module 30 includes an image signal processor 40. The image signal processor 40 receives image data from the image sensor 34, the time-of-flight sensor 36, and the inertial sensor 38. Data from the inertial sensor 38 may be routed through a serializer/deserializer 42 (described in greater detail below) outside of the camera module 30, and then transmitted back to the image signal processor 40. Alternately, data from the inertial sensor 38 is transmitted directly from the inertial sensor to the image signal processor 40, without leaving the camera module 30. Alternately, data from the inertial sensor 38 may be routed in any other suitable manner that causes that data to reach the image signal processor 40. According to other embodiments, the image signal processor 40 is located in the transceiver 50, described in greater detail below. According to other embodiments, the image signal processor 40 is located in the hub 70, described in greater detail below. According to still other embodiments, the image signal processor 40 is omitted, and a different processor at the camera module 30, transceiver 50 and/or hub 70 performs the functions that otherwise would be performed by the image signal processor 40.

Regardless of its location, the image signal processor 40 utilizes the information provided by the time-of-flight sensor 36 and the inertial sensor 38 to modify the data received from the image sensor 34 in order to reduce or eliminate shakiness in the image data received from the image sensor 34. Motion sickness can be experienced by a person who views a moving image on a screen. The more that a moving image is unstable, the greater the potential that a viewer may experience motion sickness upon viewing that moving image. Such motion sickness can result in nausea and vomiting, both of which are undesirable in a surgical setting. By integrating data from the image sensor 34, the time-of-flight sensor 36, and the inertial sensor 38 to reduce or eliminate shakiness in the moving images captured by the image sensor 34, the potential for motion sickness by a viewer is reduced or eliminated, and the image quality is enhanced. In addition, the continuous or near-continuous focusing provided by the combination of the time-of-flight sensor 36 and the liquid lens 32 causes the video experienced by a viewer to be in focus or close to in focus, further reducing the potential for a motion sickness effect that could be experienced by a viewer. The use of the liquid lens 32, the time-of-flight sensor 36, and the inertial sensor 38 in combination synergistically improves video stability and watchability.

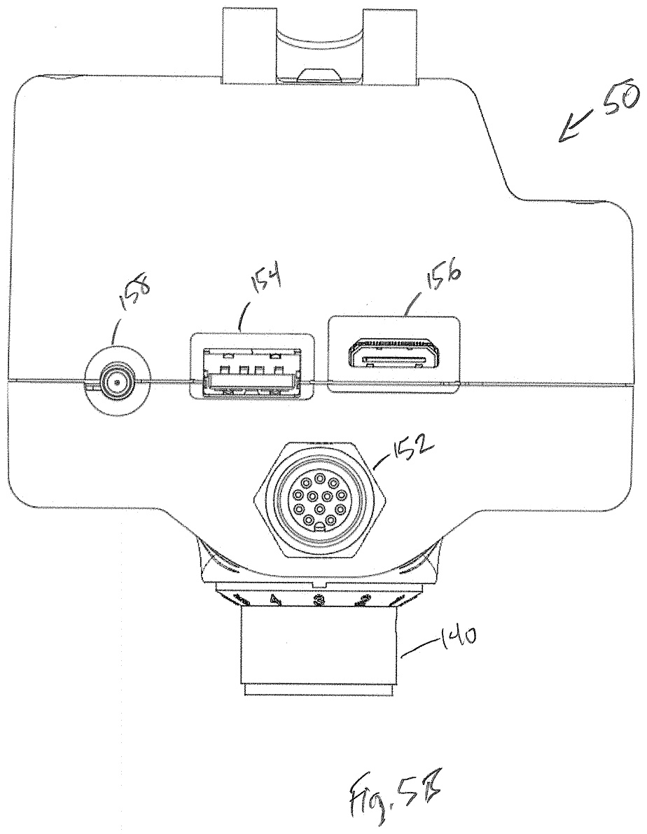

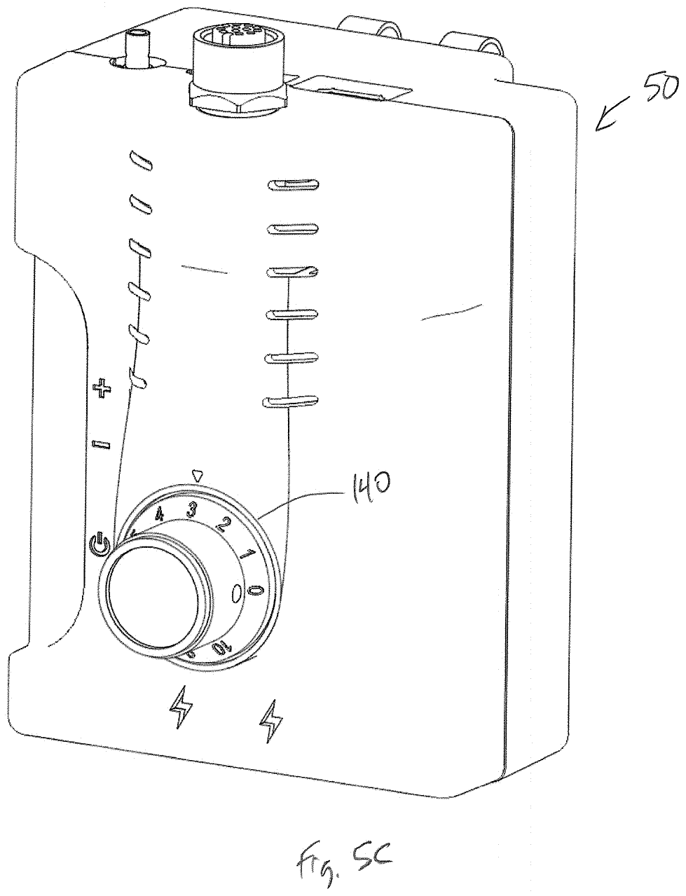

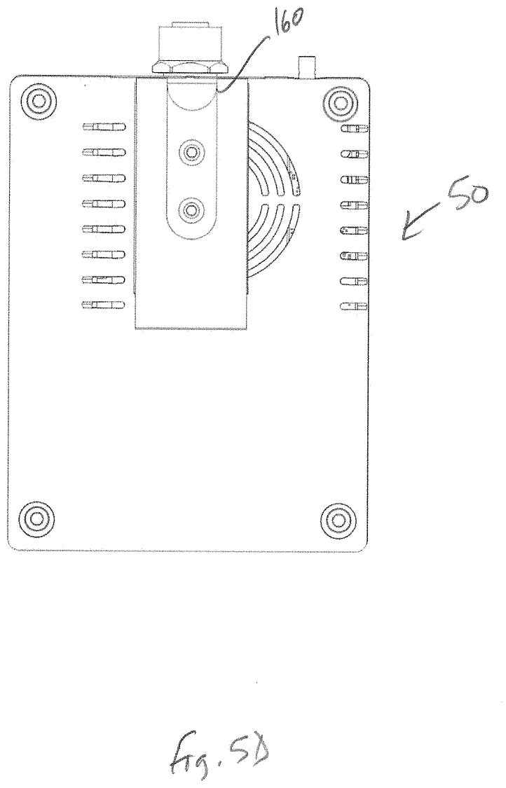

Referring to FIGS. 5A-5D, a transceiver 50 is shown. The transceiver 50 provides a data connection between the camera system 120 and the hub 70, as described in greater detail below. Optionally, the transceiver 50 includes a light control 140. The light control 140 provides the ability to control the intensity of the light emitted by the light module 20. The light control 140 may be operable by a nurse, medical technician, or other professional so that the surgeon need not touch it, because by touching it the surgeon would compromise the sterility of the surgical field. According to some embodiments, the light control 140 may be a dial, as shown in FIGS. 5A-5C. According to other embodiments, the light control 140 may be a sliding switch, one or more buttons, a keypad, or any other suitable input. Referring to FIG. 5B, the transceiver 50 may include a variety of connectors, such as a coax connector 152, a USB connector 154, an HDMI connector 156, and/or a power connector 158. Optionally, the transceiver 50 may include a clip 160 that enhances its wearability and facilitates its placement at a suitable location on the user's body.

According to some embodiments, the image signal processor 40 may output data to a serializer/deserializer 42, which in some embodiments may be located in the camera module 30. The serializer/deserializer 42 transmits data to and receives data from a transceiver 50. According to some embodiments, the serializer/deserializer 43 is connected to the transceiver 50 via a coaxial (also called coax) cable 44 and associated connectors. One coax connector may be provided in association with the camera module 30, and another coax connector may be provided in association with the transceiver 50. According to other embodiments, the serializer/deserializer 43 is connected to the transceiver 50 via a Gigabit Multimedia Serial Link (GMSL) (Maxim Integrated Products, San Jose, Calif.) cable 44 and associated connectors. One GMSL connector may be provided in association with the camera module 30, and another GMSL connector may be provided in association with the transceiver 50. The GMSL standard provides multistream support over a single cable, reducing the number of cables in the camera system 120. Further, the GMSL standard allows aggregation of different protocols in a single connection, while meeting hospital requirements for electromagnetic interference. According to other embodiments, the serializer/deserializer 43 is connected to the transceiver 50 via any other suitable cable and/or wired data transmission standard. According to other embodiments, the serializer/deserializer 43 is connected to the transceiver 50 wirelessly.

According to other embodiments, the serializer/deserializer 43 is omitted, and data is transmitted between the camera module 30 and the transceiver 50 via a USB cable and associated connectors. The USB 3.0 standard may be utilized, such that the cable and connectors are compliant with that standard. Alternately, another version of the USB standard may be utilized, such that the cable and connectors are compliant with that version of the USB standard. One USB connector may be provided in association with the camera module 30, and another USB connector may be provided in association with the transceiver 50. Regardless of the protocol used, one or more connectors 45 may be included in the camera module 30

The serializer/deserializer 42 may receive from the image signal processor 40 data that includes image data (such as in raw or Bayer format), inertial data from the inertia sensor 38, and/or time-of-flight data from the time-of-flight sensor 36, and then serialize that data for transmission to the transceiver 50. The serializer/deserializer 42 may receive from the transceiver 50 control data for the liquid lens 32 to adjust the liquid lens 32 for calibration or manual adjustments (without time of flight focus), firmware updates for the processors and sensors associated with the camera module 30, and/or other data.

According to other embodiments, the image signal processor 40 is located elsewhere than the camera module 30. In such embodiments, image data from the image sensor 34, the time-of-flight sensor 36, and the inertial sensor 38 are transmitted to the image signal processor 40 in any suitable wired or wireless manner. The components of the transceiver 50 may be distributed across two or more separate housings on the user, for balance or other considerations. One or more processors in the transceiver 50 may be distributed across two or more separate housings on the user, for balance or other considerations. Further, components described in this document as being located in the camera module 30 may instead be located in the transceiver 50, and vice versa.

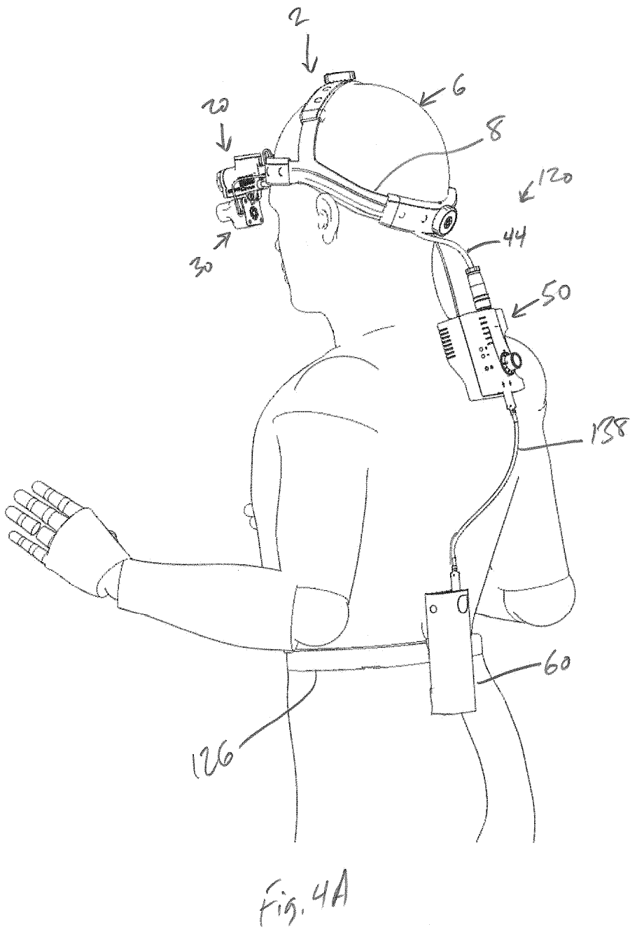

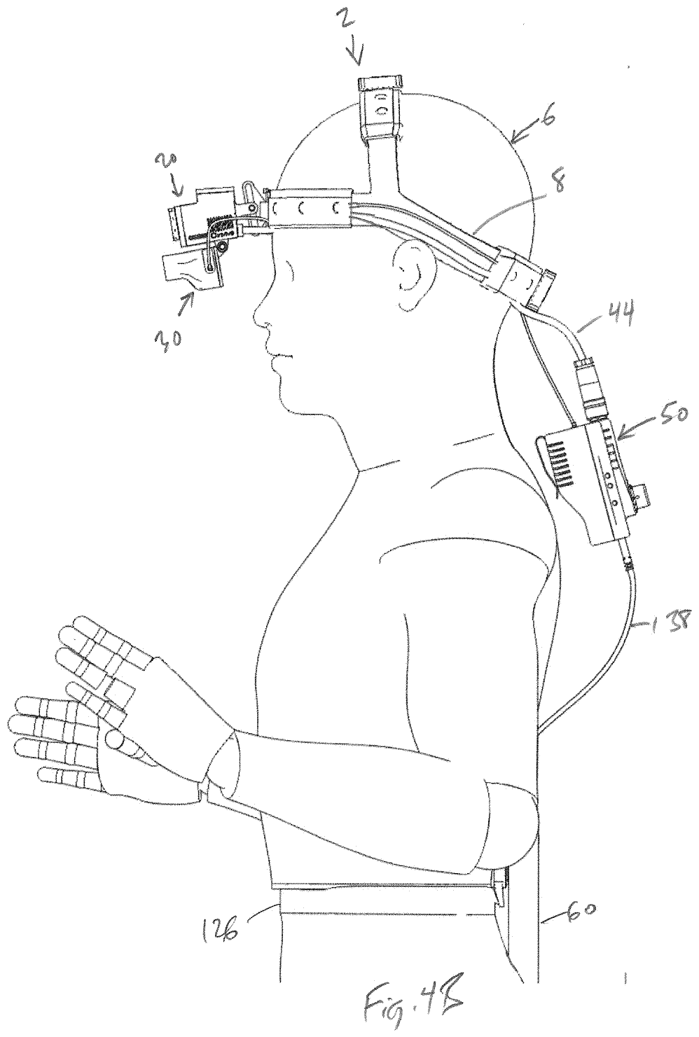

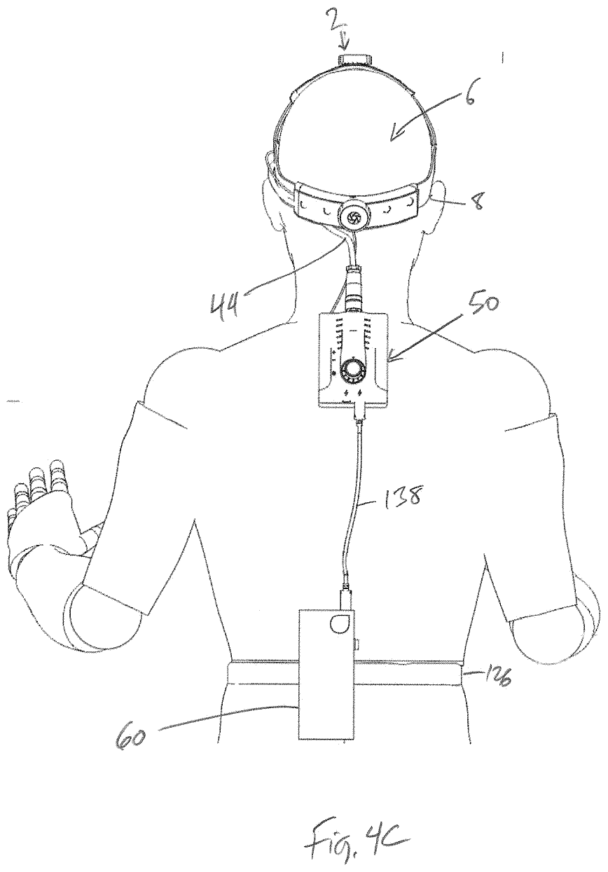

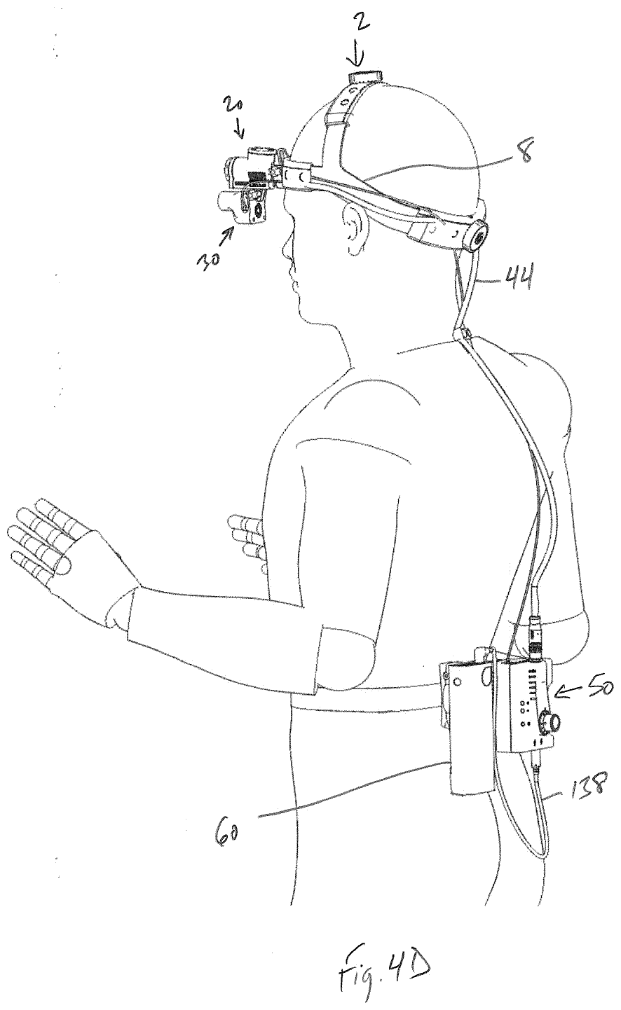

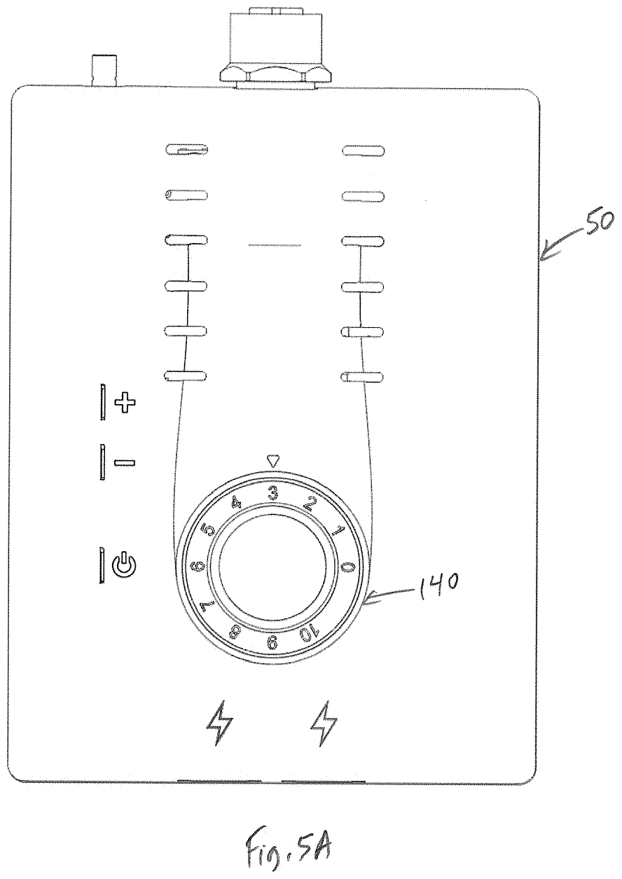

Referring also to FIGS. 4-6, an exemplary camera system 120 includes the camera assembly 2, and one or more additional body-mounted components. The body-mounted components advantageously are arranged ergonomically, to minimize and to balance the weight on the user's head 6, and to place heavier components closer to the user's waist. The serializer/deserializer 42 may be located in transceiver 50. Referring to FIGS. 4A-4C, according to some embodiments the transceiver 50 may be configured to be carried at the back of the user's neck by the shoulders, or at another location on the back. Referring to FIG. 4D, according to other embodiments, the transceiver 50 may be configured to be carried near the user's waist in the same manner as the battery 60. As another example, the transceiver 50 may be worn on a user's chest, a user's side, on an arm or leg, or at any other position on the user's body. The transceiver 50 may be secured to the user in any suitable manner and with any suitable structure or mechanism. According to some embodiments, the transceiver 50 may be attached to one or more straps, which the user may wear on his or her shoulders, to carry the transceiver 50 below the user's neck on his or her back. Alternately, if the transceiver 50 is relatively light, the transceiver 50 may hang from and be supported by the rear of the strap 8. The strap 8 may include one or more wire guides 9 that hold one or more wires 124 that carry data to and/or from the camera module 30, and that carry power to the light module 20 and camera module 30.

A battery 60 may be worn by the user. The battery 60 may be worn anywhere on the user's body and may be secured to the user's body in any suitable manner. According to some embodiments, the battery 60 may be most conveniently and comfortably placed about the user's waist or hips using a belt 126. According to other embodiments, the battery 60 may take the form of a backpack or other ergonomically-desirable configuration. Advantageously, the battery 60 is rechargeable, and easily detachable from the associated belt or other support that carries the battery 60. In this way, the battery 60 can be replaced quickly and easily with a fully-charged one if the battery 60 becomes depleted during a surgical procedure. According to other embodiments, the battery 60 is not rechargeable, or is integrated into and not detachable from the associated belt or other support.

The battery 60 is connected to one or more of the light module 20, the camera module 30 and the transceiver 50, in order to supply power thereto. According to some embodiments, the battery 60 may be connected to one or more of the light module 20, the camera module 30 and the transceiver 50 with separate, individual cables, in order to power one or more such components independently. According to other embodiments, the battery 60 may be connected directly to only one of the light module 20, the camera module 30 and the transceiver 50, and the other modules are electrically connected to the module which receives power from the battery 60. In this way, the number of power cables required by the camera system 120 may be reduced. As one example, the transceiver 50 receives power from the battery 60 via a power cable 138, and then distributes power to the light module 20, camera module 30, and any other components of the camera system 120.

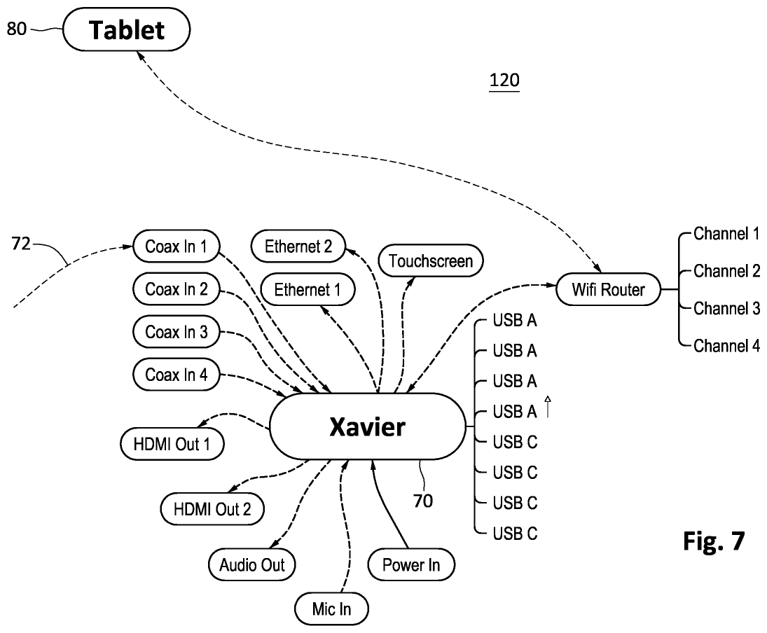

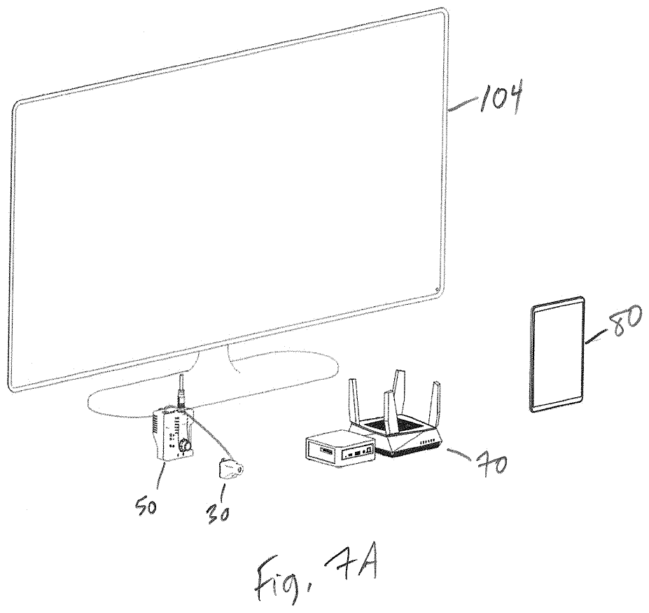

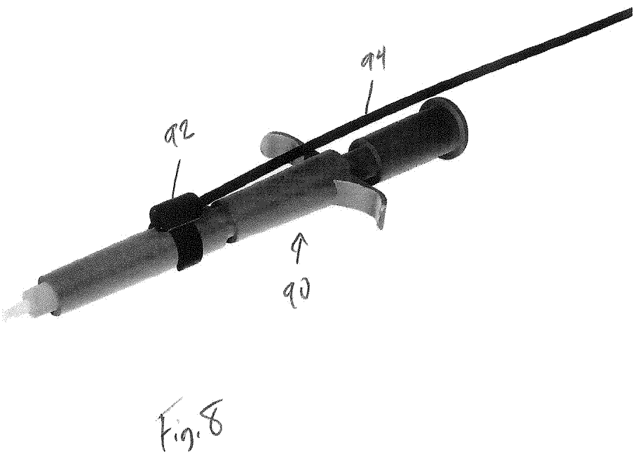

Referring also to FIGS. 7-8, the transceiver 50 has a data connection with a hub 70. The data connection between the transceiver 50 and the hub 70 may be accomplished wirelessly, such as via Wi-Fi. In this way, the user of the camera assembly 2 has greater freedom of motion and does not need to worry about becoming tangled in cable. Alternately, the data connection between the transceiver 50 and the hub 70 may be accomplished via a cable 72, such as a coax cable. The data connection between the transceiver 50 and the hub 70 may be controlled by the main processor 51, which may control the flow of data to and/or from the hub 70, as well as to and/or from the camera module 30, light module 20, and/or other head-mounted components of the camera system 120.

The hub 70 may include one or more ports for coax, HDMI, Ethernet, or other connections. Those ports may be used to receive data from other cameras or sensors, and transmit data to a network, to one or more monitors 104, or other locations. Multiple individuals in proximity to the patient may wear a camera assembly 2, and the data output from each camera assembly may be transmitted to the same hub 70, in the same manner as described above.

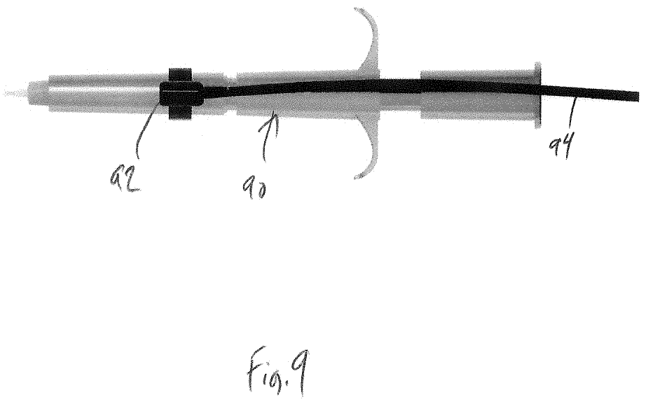

Optionally, referring also to FIGS. 8-9, a camera 92 may be attached to one or more surgical tools 90 used by a surgeon in order to improve accuracy in the use of that tool. Very small cameras are known. As one example, the Omnivision OV6948 camera (Omni Vision Technologies, Inc.; Santa Clara, Calif.) is only 0.575×0.575×0.232 mm in size. Such tiny cameras are inexpensive enough that it can be incorporated into a single-use medical device, obviating the need for sterilization between procedures. Additionally, by attaching a camera to one or more surgical tools for open surgery, the need for a separate endoscope or other relatively-bulky camera (and its support equipment) may be eliminated, reducing costs and reducing the amount of equipment needed in the operating room. A camera 92 may be attached to any surgical tool 90. For example, FIGS. 8-9 show a camera 92 attached to a standard aortic cutter 92. The inclusion of a camera 92 on the aortic cutter 90 allows the user a better view of the tissue to be treated, but also may allow the user to inspect the hole made by the aortic cutter 90 in tissue to determine whether that hole includes nicks or other abnormalities that may affect a later part of a surgical procedure. This ability to inspect closely the result of use of any surgical tool provides additional assurance that the intended procedure was performed as intended and as expected. Data from the camera 92 is transmitted to the transceiver 50 in the same manner or a similar manner as described above with regard to the camera module 30. A cable 94 may transmit data from the camera 92 to the transceiver 50. According to other embodiments, the camera 92 transmits data wirelessly to the transceiver 50. According to other embodiments, data from the camera 92 is transmitted directly to the hub 70 without utilizing the transceiver 50. According to some embodiments, data may be transmitted between the light module 20 and the hub 70 directly without utilizing the transceiver 50. According to other embodiments, a camera 92 may be attached to something other than a surgical tool 90. For example, a camera 92 may be attached to a tool or device used by an EMT or paramedic at an accident scene.

According to other embodiments, any camera that may be potentially useful for recording diagnosis and treatment of a patient, or otherwise useful in providing healthcare, may be connected directly or indirectly to the hub 70, and may be recorded and utilized like any other input to the hub 70. As one example, a camera 92 may be positioned in an ambulance to view the patient during transport. As another example, a camera 92 may be positioned in a hospital room, treatment room and/or diagnosis room. As another example, the camera 91 may be a standard body-mounted camera worn by an EMT, paramedic, firefighter or law enforcement officer.

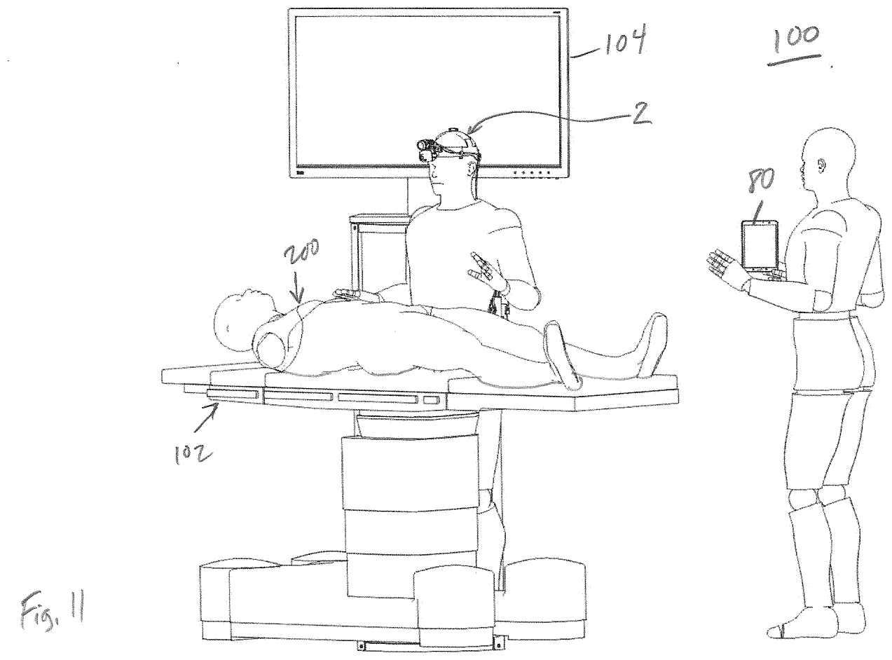

Referring also to FIGS. 6, 7A and 10-11, a tablet 80 or other device may be used to control how and where the data is output from the hub 70. The tablet 80 may be connected to the hub 70 wirelessly, such as through a Wi-Fi connection, or may be connected to the hub 70 by a cable, such as a coax or HDMI cable. The hub 70 may be located in an operating room. However, the hub 70 and other components described in this document may be used in any other location where surgery or other medical treatment is performed, such as a field hospital or treatment room.

Operation

In use, one or more users put on one or more components of the camera system 120 as described above, in particular the camera assembly 2 that is worn on the user's head. Each user may be any healthcare professional who is authorized to be in proximity to a patient, such as but not limited to a physician, nurse, medtech, EMT, paramedic, orderly, or vendor representative. The more users, the greater the flexibility of the camera system 120 and the greater the ability to switch between different views.

The camera system 120 may be utilized across a spectrum of healthcare uses and environments, such as operating rooms, catheterization labs, treatment rooms, diagnosis rooms, emergency rooms, accident sites, and locations outside of a hospital or healthcare building. An example of the use of the camera system 120 for surgery in an operating room is described below, but this example does not limit the use of the camera system 120 or the environment in which the camera system 120 may be used. During surgery, the patient 200 may be positioned on an operating table 102 in the operating room. One or more monitors 104 may be positioned in the operating room 100, whether mounted permanently to a wall or other structure, or placed on stands that may be moved. One or more monitors 104 may be placed in a location outside the operating room 100, which may be adjacent to the operating room 100, may be in the same building and spaced apart from the operating room 100, or may be in a different building from the operating room 100. The hub 70 transmits video from the camera module 30 to one or more monitors 104. A user may utilize the tablet 80 to control video transmission from the hub 70 to the one or more monitors 104. As one example, the same video transmission may be sent to every monitor 104. As another example, at least one monitor 104 receives a different video transmission from the hub 70 than at least one other monitor 104. In this way, different views of the open surgery may be shown on different monitors 104. As one example, a surgeon and an attending nurse each may wear a camera assembly 2, and a camera 92 may be attached to a surgical tool 90 used in the procedure. In this example, three separate video streams are generated, and are received by the hub 70; each of those video streams may be shown at the same time on different monitors 104. Alternately, one or two of the three video streams may be shown on one or more different monitors 104, omitting one or two of the video streams. The tablet 80 and its user may be located in the operating room 100, or in a remote location, as long as the tablet 80 has a data connection to the hub 70. According to some embodiments, the hub 70 may be configured to stream video and audio 71 via the internet or other communications network to remotely-located viewers. As used in this document, the terms “stream” and “streaming” have their conventional meaning of broadcasting a substantially continuous feed of visual and/or audiovisual data via the Internet. Such a stream 71 may be a livestream, such that interested people like medical students or physicians can view the procedure at substantially the same time as the physician performs it. The stream 71 may be one-way, in which viewers can view the stream 71 but not interact with it, or two-way, in which one or more viewers can transmit audio and/or video themselves back to the hub 70. Two-way streaming 71 may be useful where specialist knowledge of a remotely-located physician would be useful, such that the remotely-located physician can provide helpful information to the physician performing the procedure. In accordance with some embodiments, all video and audio is streamed 71 from the hub 70, and the monitor or monitors 104 receive and show a stream 71 received from the hub 70.

The lead physician initiates a procedure on the patient 200 as he or she would in the absence of the camera system 120. The physician may make one or more incisions in the patient 200 for open surgery, may make one or more incisions or openings in the patient 200 for endoscopic surgery, may make one or more incisions or openings in the patient 200 to place an access port or trocar port, may access the femoral artery or other blood vessel of the patient 200 for an percutaneous procedure, and/or may in any other clinically suitable manner, in the physician's judgment, disrupt the integrity of the patient's skin to initiate treatment. As used in this document, any and all such actions are defined as “treating the patient to access a treatment area.” The treatment area may be a surgical field. According to other embodiments, the treatment area may be an area reached endoscopically or percutaneously.

The user or users wearing one or more components of the camera system 120 acquire video of the treatment area with at least one head-mounted camera assembly 2. That video may be acquired by directly viewing the surgical field during open surgery. Where the procedure includes an endoscopic or percutaneous component, that video may be acquired from viewing the control and/or display elements associated with the endoscopic or percutaneous component of the procedure. In this way, the viewer of the video from the camera system 120 can obtain greater knowledge of the overall procedure, which may be useful from an instructional standpoint and also from the standpoint of retaining a record of the particular procedure performed on that particular patient. The user or users of the camera system 120 look wherever he, she or they would look to perform the procedure in the absence of the camera system 120. It is up to the user of the tablet 80 to select and control the video stream or streams that are output to the monitor or monitors 104 and/or streamed 71 outward by the hub.

The physician performs the procedure in the same manner that he or she would without the use of the camera system 120. As described above, if one or more users need to change the size of the spot illumination of the surgical field from the light module 20, the swappable lens 130 of the light module 20 may be changed out. Video acquired by each user's head-mounted camera assembly 2 may be stabilized by the image signal processor 40 associated with that head-mounted camera assembly 2. Then, that stabilized video is transmitted to and received by the hub 70. Another user, who may or may not be wearing one or more components of the camera system 120, controls the video output from the hub 70, such as via a tablet 80, as described above. The video output may be controlled to appear on one or more monitors 104 inside and/or outside the operating room, may be controlled to stream to recipients outside the operating room, and/or may be controlled to be saved locally or remotely.

According to some embodiments, one or more of the video streams received by the hub 70 are saved for later viewing. Such one or more video streams may be saved at the hub 70 itself, and/or on removable media associated with the hub 70. According to some embodiments, all video streams received by the hub 70 are stored. In this way, a record of the surgical procedure may be saved by the hospital, the surgeon, and/or others for legal, regulatory and/or compliance purposes. The saved videos may be saved in a system that allows for access by other doctors, medical students, or the public, for learning and educational purposes. The saved videos may be streamed at a later time, or on-demand. Such video storage and streaming may be particularly useful for medical students at a time such as during the COVID-19 pandemic, in which in-person learning may be limited or suspended altogether.

While the example above describes the use of a camera system 120 in an operating room, a user may utilize the camera system 120 in any other suitable location. Regardless of the particular location, the camera system 120 functions substantially as described above. For example, the camera system may be used in a hospital room, a treatment room, a field hospital, an emergency room, in the field at an accident site, or any other suitable location. Further, the user may utilize the camera system 120 in conjunction with any medical evaluation or treatment. As one example of this, a physician may utilize a fluoroscope in the course of cardiac catheterization. In the course of performing this procedure, while utilizing the camera system 120, the physician may perform several actions, including puncturing the patient's femoral artery, inserting a guidewire, and viewing a fluoroscope. The camera module 30 captures video of what the physician is looking at during the procedure. The fluoroscope may be connected to the hub 70, so that the hub 70 receives video from the fluoroscope.

As another example, a physician and/or nurse may utilize the camera system 120 in a treatment room at a doctor's office or hospital, in the course of diagnosing and/or treating a patient. During use of the camera system 120, no surgical treatment or other treatment that disrupts the integrity of a patient's skin need be performed. Instead, the physician may simply diagnose the patient.

As another example, the camera system 120 may be utilized by an EMT or paramedic at an accident site. The hub 70 may be located in an ambulance, and may be capable of transmitting video and other data via any suitable communication technology, such as cellular network data service. When used by an EMT or paramedic at an accident site or other site where emergency treatment of a patient is necessary, two-way streaming 71 may be useful, because such two-way streaming would allow a remotely-located doctor to provide instructions to the EMT or paramedic based on the content of the stream 71.

As used in this document, and as customarily used in the art, terms of approximation, including the words “substantially” and “about,” are defined to mean normal variations in the dimensions and other properties of finished goods that result from manufacturing tolerances and other manufacturing imprecisions, and the normal variations in the measurement of such dimensions and other properties of finished goods.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

7. The camera system of claim 1, further comprising a transceiver connected to said camera module.

8. The camera system of claim 7, further comprising a cable, wherein said transceiver is connected to said camera module through said cable.

9. The camera system of claim 1, wherein said camera module further comprises a serializer/deserializer, and wherein said image signal processor is connected to said transceiver through said serializer/deserializer.

10. The camera system of claim 1, wherein said image signal processor is connected to said hub via said transceiver.

11. The camera system of claim 10, wherein said transceiver is wirelessly connected to said hub.

12. The camera system of claim 1, wherein said data connection between at least one said camera assembly and said hub is wireless.

13. The camera system of claim 1, further comprising a surgical tool and at least one camera attached to said surgical tool, wherein said camera is wirelessly connected to said hub.

14. The camera system of claim 1, wherein said image signal processor is located in said camera module.

15. A method for utilizing a camera system in conjunction with providing healthcare to a patient by at least one user, comprising:
placing a head-mounted camera assembly onto at least one user;
acquiring video with each said head-mounted camera assembly;
transmitting said video from each said head-mounted camera assembly to a hub;
receiving said video at said hub; and
controlling video output from said hub.

16. The method of claim 15, further comprising stabilizing said video at said hub.

17. The method of claim 16, further comprising storing said video after said stabilizing.

18. The method of claim 15, wherein said controlling comprises streaming said video output.

19. The method of claim 15, further comprising performing said acquiring in an operating room.

20. The method of claim 19, further comprising making an incision in the patient to reveal a surgical field; and
wherein said acquiring comprises acquiring video of the surgical field.
